(19) United States
(12) Patent Application Publication
(10) Pub. No.: US 2005/0214237 A1
(43) Pub. Date: Sep. 29, 2005

(54) Agent and method for permanent hair deformation based on organic borohysride derivatives

(76) Inventors: Thilo Arlt, Olefingen (CH); Peirre Alain Clement, Murten (CH)

Correspondence Address: Striker Striker & Stenby, 103 East Neck Road, Huntington, NY, 11743, US

(21) Appl. No.: 10/513,185

(22) PCT Filed: Sep. 17, 2003
(86) PCT No.: PCT/EP03/10317

§ 371 Date: Nov. 1, 2004

(30) Foreign Application Priority Data
Nov. 2, 2002 (DE) 102 51 104.7

(51) Int. Cl. A61K 7/09

(52) U.S. Cl. 424/70.2

(57) ABSTRACT

The invention relates to an agent for permanently shaping hair that contains, as a keratin-reducing active substance, a tetraalkylammonium boron hydride and/or a boron alkoxyhydride, and relates to a method for permanently shaping hair while using agents of the aforementioned type.

The present invention relates to an agent for permanent hair deformation containing as the keratin-reducing constituent certain organic borohydride derivatives and to a method for permanent hair deformation that uses such an agent.

As is known, conventional technology for carrying out permanent hair deformation is based on two treatment steps. In the first step, the cystine disulfide bridges of hair keratin are opened by the action of an agent containing a reducing constituent (deformation agent). The hair is then set to the desired shape. In a second step, the cystine disulfide bonds are again closed by use of a fixing agent, namely an agent containing an oxidant.

The conventional permanent wave reducing agent used thus far has been thioglycolic acid, for example in the form of its ammonium or monoethanolamine salt. Other common hair keratin-reducing agents are inorganic sulfites, 2-mercaptopropionic acid (thiolactic acid), 3-mercaptopropionic acid, certain mercaptocarboxylate esters, cysteine and the derivatives of these compounds.

All these agents, however, present a number of drawbacks. Alkaline preparations based on mercapto-carboxylic acids, although sufficiently active, cause hair damage manifesting itself, for example, in the form of extensively appearing hair breakage. In many cases, said agents also have an undesirable effect on the scalp.

Moreover, the unpleasant odor of the reducing agents employed requires an intensive use of perfume in the products. Some of the said problems can be solved by use of 2-mercaptopropionic acid (thiolactic acid). Compared to the generally used thioglycolic acid, however, thiolactic acid gives weaker deformation.

The mercaptocarboxylate esters with which hair deformation can also be achieved at lower pH values are not satisfactory in terms of skin compatibility and the risk of causing sensitization. In place of the mercapto-carboxylate esters, mercaptocarboxamides such as thioglycolamide or alkyl-substituted or hydroxyalkyl-substituted amides have also been used. These substances, like the carboxylate esters, have a high deformation potential even at low pH, but in terms of sensitization are even more critical than the esters.

To avoid some drawbacks of the thiols, attempts have also been made in the past to use a borohydride as the keratin-reducing substance. The use of alkali metal borohydrides for the permanent deformation of hair has been known since 1956, for example from U.S. Pat. No. 2,766,760.

Different approaches have been taken to reduce the reactivity of alkali metal borohydrides. For example, a method is known that involves embedding the hygroscopic and flammable alkali metal borohydrides in solid and pasty inert embedding agents (German Published Patent Application DE-AS 1,069,588) or in a carrier consisting of synthetic wax (GB Patent 835,247). Encapsulation of sodium borohydride or potassium borohydride in a coating of an ethylene oxide polycondensation product, a polyoxyethylated fatty alcohol or a fatty alcohol partly converted by sulfuric acid (DE-AS 1,199,927) has also been tried for the purpose of achieving stabilization.

Also known is a method for permanent deformation of hair with sodium borohydride or potassium borohydride wherein the solvent constitutes an aqueous, aqueous-alcoholic or alcoholic solution (DE-AS 1,129,657).

A common feature of all methods using alkali metal borohydrides, however, is that the solution must be prepared just before use, because complete stabilization of sodium and potassium borohydride in water or alcohol could not be achieved. In the agent of the invention, this drawback of the prior art was eliminated by using borohydrides that are soluble and stable in alcoholic solution, meaning that they do not evolve hydrogen and, hence, are also not flammable. This obviates the need for preparing fresh borohydride-containing solutions just before use.

The object of the present invention is therefore an agent for permanent deformation of hair, characterized in that it contains
a) a tetraalkylammonium borohydride of general formula
   N(R₁R₂R₃R₄)⁺BH₄⁻ (I)
   wherein the R₁ to R₄ groups independently of each other denote a straight-chain or branched alkyl group with 1 to 10 carbon atoms or an alkylaryl group with 5 or 6 ring atoms, with the provision that not all groups are simultaneously methyl groups, or
b) a boralkoxy hydride of general formula 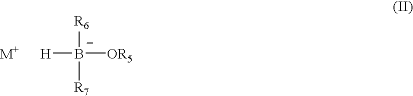
   wherein R₅ denotes a straight-chain or branched alkyl group with 1 to 6 carbon atoms, R₆ and R₇ independently of each other denote H, halogen, a straight-chain or branched alkyl or alkoxy group with 1 to 6 carbon atoms and M⁺ is any inorganic or organic cation, preferably sodium, potassium or ammonium.

Preferred compounds of formula (I) are those wherein R₁ to R₄ independently of each other denote —CH₃, —CH₂CH₃, —CH₂CH₂CH₃, —CH₂CH₂CH₂CH₃, —CH(CH₃)₂, —CH₂CH₂CH₂N(CH₃)₂, —CH₂CH₂OCH₃, —CH₂CH₂CH₂OCH₃, or benzyl.

Particularly preferred compounds of formula (I) are those wherein at least one of the R₁ to R₄ alkyl groups has a chain length of at least two carbon atoms.

Especially preferred compounds of formula (I) are tetraethylammonium borohydride, tetrapropylammonium borohydride, tetrabutylammonium borohydride and benzyltriethyl-ammonium borohydride.

Preferred compounds of formula (II) are those wherein all substituents are equal and denote an alkoxy group with 1 to 6 carbon atoms.

Particularly preferred compounds of formula (II) are sodium trimethoxyborohydride and potassium triisopropoxyborohydride.

The borohydrides of general formula (I) or (II) are used in the ready-for-use agent for hair deformation in a total amount from 1 to 20 weight percent and preferably from 5 to 15 weight percent.

In another embodiment of the invention, the borohydrides present in the agent of the invention can also be used in admixture with at least one known thiol such as thioglycolic acid, thiolactic acid, cysteine, cysteamine, alkylcysteamine or acylcysteamine or a sulfite.

The deformation agent can be sold as a one-component or two-component package. In the case of a two-component package, the two components are mixed with one another just before use. The agent can then be in the form of an aqueous, aqueous-alcoholic or alcoholic solution or of an emulsion or in a thickened water-based form, particularly as a cream, gel, foam or paste. A one-component agent is preferably a solution or a gel based on an alcohol with 1 to 6 carbon atoms. Preferred alcohols are ethanol, 1-propanol, isopropanol, 1-butanol, isobutanol, 1-pentanol and 1-hexanol. Solutions or gels in propylene glycol, a polyglycol, glycerol or tetrahydrofuran are also suitable.

The deformation agent can, of course, also contain all known additives usually added to such agents, for example thickeners such as bentonite, fatty acids, starch, polyacrylic acid and the derivatives thereof, cellulose derivatives, alginates, vaselines, paraffin oils; wetting agents or emulsifiers from the classes of anionic, cationic, amphoteric or nonionic surface-active agents, for example fatty alcohol sulfates, fatty alcohol ether sulfates, alkylsulfonates, alkylbenzenesulfonates, quaternary ammonium salts, alkylbetaines, ethoxylated alkylphenols, fatty alkanolamides or ethoxylated fatty acid esters; furthermore opacifiers, for example polyethylene glycol esters; alcohols, for example ethanol, propanol, isopropanol and glycerol; sugars, for example D-glucose; dissolution promoters, stabilizers, buffering agents, perfume oils, dyes and hair-conditioning and hair-care constituents, for example cationic polymers, lanolin derivatives, cholesterol, pantothenic acid and betaine.

The said constituents are used in amounts usually employed for such purposes, for example the wetting agents and emulsifiers at a total concentration of 0.2 to 30 weight percent, the alcohols in a total amount from 0.1 to 20 weight percent, the opacifiers, perfume oils and dyes in an amount from 0.01 to 1 weight percent each, the buffering agents in a total amount from 0.1 to 10 weight percent, sugars, dissolution promoters, stabilizers, hair conditioners and hair-care constituents in an amount from 0.1 to 5 weight percent each, the thickeners and dissolution promoters possibly being present in a total amount from 0.5 to 20 weight percent.

Moreover, to boost the activity of said agent, it is possible to add a swelling agent and a penetrant, for example dipropylene glycol monomethyl ether, 2-pyrrolidone or 2-imidazolidinone, in an amount from 1 to 30 weight percent, and for purposes of preventing excessive hair curling a dithio compound, for example dithiodiglycolic acid, dithiolactic acid or a salt of such a dithiol.

By varying the concentration, it is possible to provide an agent suitable for use on any hair structure, optionally by additional application of heat. The agent brings about an elastic, durable and uniform deformation from the roots to the tips of the hair.

Furthermore, the present invention relates to a method for permanent hair deformation whereby the hair, before and/or after it is brought into the desired shape, is treated with a deformation agent, rinsed with water, then treated oxidatively, rinsed with water, optionally set to a wave with water and then dried, said method being characterized in that the deformation agent used is the afore-described agent of the invention.

In a preferred embodiment of the method of the invention, the hair is first washed with a shampoo and then rinsed with water. The towel-dried hair is then divided into individual strands and wound around curlers having a diameter from 5 to 30 millimeters and preferably from 5 to 15 millimeters. The hair is then treated with an amount of the deformation agent of the invention sufficient for hair deformation, preferably an amount from 60 to 120 grams.

After an exposure time sufficient for permanent deformation of the hair which amounts to from 5 to 30 minutes (10 to 30 minutes without applying heat; 5 to 20 minutes with heat application), depending on the hair structure, the pH and the deformation efficacy of the deformation agent and on the application temperature, the hair is rinsed with water and then oxidatively post-treated (“fixed”). Depending on the hair fullness, the post-treatment agent is preferably used in an amount from 80 to 100 grams.

For the oxidative post-treatment in the wound or unwound state, any post-treatment agent suitable for this purpose can be used. For example, the oxidants that can be employed in such post-treatment agents are potassium bromate, sodium bromate, sodium perborate, urea peroxide and hydrogen peroxide. The concentration of the oxidant varies depending on the application time (as a rule 5 to 15 minutes) and the application temperature. Normally, the oxidant is present in the ready-for-use aqueous post-treatment agent at a concentration from 0.5 to 10 weight percent. The agent for the oxidative post-treatment can, of course, also contain other substances, for example wetting agents, hair-care agents, such as cationic polymers, weak acids, buffering agents or peroxide stabilizers and be in the form of an aqueous solution or an emulsion or in thickened, water-based form, particularly a cream, gel or paste. In particular, these common additives can be present in the post-treatment agent in an amount of 0.1 to 10 wt. %.

The curlers are then removed. If necessary, the unwound hair is once again subjected to oxidative post-treatment after which it is rinsed with water, optionally set to a wave with water and finally dried.

The following examples will explain the subject matter of the invention in greater detail.

### One-Component Permanent Deformation Agent

### Example 1

### Permanent Wave Agent for Normal Hair

| | | |
|---|---|---|
| 7.6 | g | of tetraethylammonium borohydride |
| 0.5 | g | of ethoxylated polydimethylsiloxane (12 EO) |
| | | (CTFA: PEG-12 DIMETHICONE) |
| 91.9 | g | of ethanol |

| | |
|---|---|
| 100.0 | g |

Normal, not previously damaged hair was washed, rubbed with a towel and wound onto curlers having a diameter of 6 millimeters. The hair was then uniformly moistened with the afore-described hair deformation agent. After an exposure time of 15 minutes, the hair was thoroughly rinsed with water and then post-treated oxidatively with 80 grams of a 3-percent aqueous hydrogen peroxide solution. After removing the curlers, the hair was again rinsed with water, set to a wave with water and then dried. The hair thus treated had uniform and lively curls.

### Example 2

### Permanent Deformation Agent for Normal Hair

| | | |
|---|---|---|
| 12.8 | g | of tetrabutylammonium borohydride |
| 0.1 | g | of perfume |
| 87.1 | g | of 1-butanol |
| 100.0 | g | |

Normal, not previously damaged hair was washed, rubbed with a towel and wound onto curlers having a diameter of 6 millimeters. The hair was then uniformly moistened with the afore-described hair deformation agent. After an exposure time of 15-25 minutes, the hair was thoroughly rinsed with water and then post-treated oxidatively with 80 grams of a 3-percent aqueous hydrogen peroxide solution. After removing the curlers, the hair was again rinsed with water, set to a wave with water and then dried. The hair thus treated had uniform and lively curls.

### Example 3

### Permanent Wave Agent for Normal Hair

| | | |
|---|---|---|
| 10.8 | g | of benzyltriethylammonium borohydride |
| 0.5 | g | of ethoxylated glycerol monococo |
| | | fatty acid ester (7 EO) |
| | | (CTFA: PEG-7 GLYCERYLCOCOATE) |
| 88.7 | g | of isopropanol |
| 100.0 | g | |

Normal, not previously damaged hair was washed, rubbed with a towel and wound onto curlers having a diameter of 6 millimeters. The hair was then uniformly moistened with the afore-described hair deformation agent. After an exposure time of 15-25 minutes, the hair was thoroughly rinsed with water and then post-treated oxidatively with 80 grams of a 3-percent aqueous hydrogen peroxide solution. After removing the curlers, the hair was again rinsed with water, set to a wave with water and then dried. The hair thus treated had uniform and lively curls.

### Two-Component Permanent Deformation Agent

### Example 4

### Permanent Deformation Agent for Normal Hair

| Component A | | |
|---|---|---|
| 12.8 | g | of tetraethylammonium borohydride |
| Component B | | |
| 1.0 | g | of dimethyldiallylammonium chloride |
| | | homopolymer (CTFA: POLYQUATERNIUM-6) |
| 86.2 | g | of water |
| 87.2 | g | |

Just before use, the above two components A and B were mixed to form the ready-for-use hair deformation agent. Normal, not previously damaged hair was washed, rubbed with a towel and wound onto curlers having a diameter of 6 millimeters. The hair was then uniformly moistened with the ready-for-use hair deformation agent. After an exposure time of 15-25 minutes, the hair was thoroughly rinsed with water and then post-treated oxidatively with 80 grams of a 3-percent aqueous hydrogen peroxide solution. After removing the curlers, the hair was again rinsed with water, set to a wave with water and then dried. The hair thus treated had uniform and lively curls.

### Example 5

### Permanent Deformation Agent for Normal Hair

| Component A | | |
|---|---|---|
| 6.4 | g | of sodium trimethoxyborohydride |
| Component B | | |
| 1.0 | g | of polyethylene glycol (8 EO) (CTFA: PEG-8) |
| 92.6 | g | of 1-pentanol |
| 93.6 | g | |

Just before use, the above two components A and B were mixed to give the ready-for-use hair deformation agent. Normal, not previously damaged hair was washed, rubbed with a towel and wound onto curlers having a diameter of 6 millimeters. The hair was then uniformly moistened with the afore-described hair deformation agent. After an exposure time of 15-25 minutes, the hair was thoroughly rinsed with water and then post-treated oxidatively with 80 grams of a 3-percent aqueous hydrogen peroxide solution. After removing the curlers, the hair was again rinsed with water, set to a wave with water and then dried. The hair thus treated had uniform and lively curls.

1. Agent for permanent deformation of hair, characterized in that it contains as the keratin-reducing agent
a) a tetraalkylammonium borohydride of general formula
N(R₁R₂R₃R₄)⁺BH₄⁻ (I)
wherein the R₁ to R₄ groups independently of each other denote a straight-chain or branched alkyl group with 1 to 10 carbon atoms or an alkylaryl group with 5 or 6 ring atoms, with the provision that not all groups are simultaneously methyl groups, or
b) a boralkoxy hydride of general formula 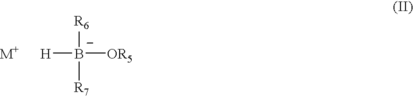
wherein R₅ denotes a straight-chain or branched alkyl group with 1 to 6 carbon atoms, R₆ and R₇ independently of each other denote H, halogen, a straight-chain or branched alkyl or alkoxy group with 1 to 6 carbon atoms and M⁺ is a cation.

2. Agent according to claim 1, characterized in that in formula (I) R₁ to R₄ independently of each other stand for —CH₃, —CH₂CH₃, —CH₂CH₂CH₃, —CH₂CH₂CH₂CH₃, —CH(CH₃)₂, CH₂CH₂CH₂N(CH₃)₂, —CH₂CH₂OCH₃, —CH₂CH₂CH₂OCH₃, or benzyl.

3. Agent according to claim 1 or 2, characterized in that the compounds of formula (I) are selected from among tetraethylammonium borohydride, tetrapropylammonium borohydride, tetrabutylammonium borohydride and benzyltriethylammonium borohydride.

4. Agent according to claim 1, characterized in that in formula (I) all substituents are equal and denote an alkoxy group with 1 to 6 carbon atoms.

5. Agent according to one of claims 1 or 4, characterized in that the compound of formula (II) is selected from among sodium trimethoxyborohydride and potassium triisopropoxyborohydride.

6. Agent according to one of claims 1 to 5, characterized in that the compound of formula (I) and/or (II) is contained in the ready-for-use agent in an amount from 1 to 20 weight percent.

7. Agent according to one of claims 1 to 6, characterized in that it contains additionally at least one hair keratin-reducing compound selected from among thioglycolic acid, thiolactic acid, cysteine, cysteamine, alkyl- or acylcysteamine or sulfites.

8. Agent according to one of claims 1 to 7, characterized in that it contains additionally at least one dithio compound.

9. Agent according to one of claims 1 to 8, characterized in that it is in the form of an alcoholic solution or a gel.

10. Method for permanent deformation of hair whereby the hair, before and/or after it has been held in the desired shape, is treated with a deformation agent, rinsed with water, oxidatively post-treated, again rinsed with water and optionally set to a wave with water and then dried, characterized in that the deformation agent used is one of the agents according to claims 1 to 9.
